(19) United States
(12) Patent Application Publication
(10) Pub. No.: US 2012/0100077 A1
(43) Pub. Date: Apr. 26, 2012

(54) MIXTURE OF AMPHIPATHIC MOLECULES AND METHOD FOR MODIFYING CELL MEMBRANES BY MEANS OF FUSION

(75) Inventors: Bernd Hoffmann, Juelich (DE); Agnes Csiszar, Niederzier (DE); Nils Hersch, Moenchengladbach (DE); Rudolf Merkel, Juelich (DE)

(73) Assignee: FORSCHUNGSZENTRUM JUELICH GMBH, JUELICH (DE)

(21) Appl. No.: 13/261,122

(22) PCT Filed: Jul. 8, 2010
(86) PCT No.: PCT/DE2010/000811

§ 371 Date: Jan. 5, 2012

(30) Foreign Application Priority Data
Jul. 9, 2009 (DE) 10 2009 032 658.8

(51) Int. Cl. A61K 49/00; A61K 38/19; C12N 5/071; A61K 47/24; A61K 47/30; A61K 38/16; A61K 48/00; A61K 33/06; A61K 31/616; A61K 31/575; A61K 31/573; A61K 31/59; A61K 31/07; A61K 31/355; A61K 31/122; C12Q 1/02; A61P 35/00; A61K 31/231; A61K 9/127

(52) U.S. Cl. 424/9.6; 424/450; 424/85.1; 435/325; 424/9.1; 514/785; 514/772; 514/1.1; 514/44 R; 424/682; 514/162; 514/182; 514/179; 514/167; 514/725; 514/458; 514/682; 435/29; 435/375; 514/547

(57) ABSTRACT

Disclosed is a mixture of amphipathic molecules and a method for modifying cells in vivo by way of membrane fusion with these molecules.

The invention relates to a mixture of amphipathic molecules and a method for modifying cell membranes by way of fusion with these molecules.

### STATE OF THE ART

The publication by Simberg et al. (Simberg D., Weisman S., Talmon, Y, Barenholz Y. (2004) DOTAP (and Other Cationic Lipids): Chemistry Biophysics, and Transfection. Crit. Reviews in Therap. Drug Carr. Systems, 2004, 21, 257-317.) discloses that cationic lipids such as DOTAP, for example, mixed with other phospholipids such as DOPE, for example, (S. W. Hui, M. Langner, Y. Zhao, P. Ross, E. Hurley, and K. Chan (1996) The Role of Helper Lipids in Cationic Liposome-Mediated Gene Transfer. Biophysical Journal, 1996, 71, 590-599.) can be used to incorporate DNA into cells (transfection). The method by which these particles are taken up by cells is endocytosis, as disclosed by Weijun et al. (Weijun Li and Francis C. Szoka Jr. 2007. Lipid-based Nanoparticles for Nucleic Acid Delivery. Pharmaceutical Research, 24, 438-449.). This method is shown to be particularly inefficient since only 1 to 10% of the DNA enclosed in liposomes is taken up by the cells.

A modification of these mixtures with further proteins such as transferrin, for example, is known from Sakaguchi et al. (N. Sakaguchi, Ch. Kojima, A. Harada, K. Koiwai and K. Kono, 2008. The correlation between fusion capability and transfection activity in hybrid complexews of lipoplexes and pH-sensitive liposomes. Biomaterials 29, 4029-4036).

A further modification of a fusion mixture is achieved by binding viral components on the surface of the carrier particles. The disadvantages of this method are the effort and cost required to produce the complexes. A further fusion system is known from Gopalakrishnan et al. (G. Gopalakrishnan, C. Danelon, P. Izewska, M. Prummer, P.-Y. Bolinger, I. Geissbühler, D. Demurtas, J. Dubochet, and H. Vogel (2006) Multifunctional Lipid/Quantum Dot Hybrid Nanocontainers for Controlled Targeting of Live Cells. Angew. Chem. Int. Ed. 2006, 45, 5478-5483.). The authors describe the fusion of a living cell membrane with a vesicular structure made of DMPE, DOTAP, DPPE-PEG2000 and Quantum-Nanodots. The disadvantage of the method is that non-biodegradable particles (Quantum-Nanodots) are used. Further medical or pharmaceutical applications are therefore ruled out.

The methods according to the prior art are therefore disadvantageously inefficient or place great stress on the cells. In addition, they must be optimized for every cell type and are therefore labor-intensive and cost-intensive.

### OBJECT AND SOLUTION OF THE INVENTION

The object of the invention therefore is to provide a mixture of molecules, which also results in efficient fusion with cell membranes in vivo. A further object of the invention is to provide a method for modifying cell membranes in vivo by way of fusion with a mixture of molecules. A further object of the invention is to demonstrate possible applications of the mixture of molecules in the modification of cells.

The object is achieved by a mixture according to claim 1 and by the method for in vivo cell (membrane) modification and by the cells provided in this manner according to alternative independent claims. The method is based on membrane fusion between an arbitrary cell membrane and a mixture of amphipathic molecules according to the invention. Biologically or pharmaceutically relevant molecules can thereby be incorporated into the cell interior and/or into the cell membrane by way of fusion and/or can be coupled thereto on the surface thereof. The mixture can also be used on single cells and, advantageously, on tissues or tissue sections.

### The Fusion Mixture

The mixture of amphipathic molecules comprises an amphipathic molecule type A which has a positive total charge in the hydrophilic region, and an amphipathic molecule type B which forms a delocalized electron system in the hydrophilic region and/or in the hydrophobic region.

A molecule type B having a delocalized electron system therefore includes, according to the invention, at least one (or several) cyclic structural motif(s) formed by conjugated double bonds and/or free electron pairs and/or unoccupied p orbitals. They follow the Hückel rule.

Particularly advantageously, covalently bound adjacent atoms of molecule type B in the delocalized electron system have an electronegativity difference (Δ_{χ}) greater than or equal to 0.4. A molecule type B that meets these rules increases the fusion efficiency.

It is furthermore advantageous that a group R forming the delocalized electron system is bound at the hydrophobic or hydrophilic region in the molecule type B.

In one embodiment of the invention, the mixture comprises 1,2-dioleoyl-3-trimethylammonium-propane (DOTAP) as molecule type A.

In a further embodiment of the invention, molecule type B is formed by a phospholipid to which a fluorescent dye having a delocalized electron system is bound. A different dye, such as an azo dye, can however be bound to a phospholipid.

In particular, 1-hexadecanoyl-2-dodecanoyl-sn-glycero-3-phosphocholine or 1,2-dihexadecanoyl-sn-glycero-3-phosphoethanolamine, or 1,2-dioleoyl-sn-glycero-3-phosphoethanolamine is provided in the mixture as the phospholipid of molecule type B.

The delocalized electron system in molecule type B is formed, advantageously, by an R group as indicated below: 2-(4,4-difluoro-5-methyl-4-bora-3a,4a-diaza-s-indacene-3-dodecanoyl) or N-(4,4-difluoro-5,7-dimethyl-4-bora-3a,4a-diaza-s-indacene-3-propionyl) or Lissamine rhodamine B sulfonyl or (5-(chlorosulfonyl)-2-(1H, 2H, 3H, 5H, 6H, 7H, 11H, 12H, 13H, 15H, 16H, 17H-pyrido[3,2,1-ij]quinolizino[1′,9′: 6,7,8]chromeno[2,3-f]quinolin-4-ium-9-yl)benzene sulphonate or 7-nitro-2-1,3-benzoxadiazol-4-yl or carboxyfluorescein or 1-pyrenesulfonyl.

The listed components of molecule type B, that is the phospholipids and the aforementioned R groups, for example, can be freely combined with one another, that is, any of the dyes can be bound to a phospholipid.

The fusion mixture has far-reaching advantages over the previously known fusion mixtures. For example, the mixture can be used with all (mammalian) cell types, i.e. ubiquitously. The mixture is highly efficient (>50%), and no damage occurs to the cells as a result of the fusion. The cells are therefore fully capable of dividing after fusion. The mixture can also be used on tissue sections. Further advantages are achieved by way of the functionalization of the cell membrane with specific molecules, as indicated below for example in method 3.

In the mixture according to the invention, a further amphipathic molecule type C can be provided as a helper molecule.

To this end, 1,2-dioleoyl-sn-glycero-3-phosphoethanolamine (DOPE) can notably be provided as molecule type C.

The preferred ratio between molecule types A, B and C is approximately 1:0.05 to 0.2:1 wt/wt.

The mixture comprises up to 99 wt % amphipathic molecule type A, 40 wt % amphipathic molecule type B (with group R), and up to 70 wt % amphipathic molecule type C.

For the method according to the invention, arbitrary mixtures are dissolved in an organic solvent and thoroughly homogenized (e.g., vortex, ultrasound). The solvent is removed, e.g. in a vacuum, and the dried mixture of molecule types is dissolved in an aqueous buffer. The mixture is permanently stable when stored under cooling. Particularly preferably, the molecule types A, B and C are present as lipids. As a result, they form a liposome when taken up in an aqueous buffer. Liposomes are preferred since further molecule types can be arranged in and on the cell membrane in an excellent manner, and molecules enclosed in the liposome can be incorporated into the cell interior during fusion of the liposome with the cell membrane.

Very particularly preferably, the mixture is therefore in the form of a liposome. The liposome according to the invention makes it possible, particularly advantageously, to arrange further molecules therein, which can result in preferred modifications of the cells.

It is also feasible, however, to use polymers in place of lipids as molecule type A and/or B and/or C.

For the method according to the invention for in vivo cell modification, the selected cell, with the cell membrane thereof, is brought into contact with a preferably buffered mixture or liposome. The procedure for establishing contact advantageously takes only a few minutes, e.g. 1 to 120, and preferably 10 to 30 minutes, during fusion. Advantageously, single cells are fused with the mixture or the liposome quickly after contact therewith is established. Cell tissues may require a somewhat longer period of time, i.e. up to approximately 120 minutes. The endocytosis of the liposome must be strictly distinguished from the fusion.

Compared to the prior art, and for the shortness of the contact time, the method results in a high portion of cells fused with the mixture or the liposome. The efficiency is therefore advantageously greater than 50%.

Various further molecules can be added to the mixture according to the invention while the method is underway. The method according to the invention is shown to be versatile and robust in this regard. By this it is meant that a multiplicity of different functional molecule types can be admixed individually or simultaneously, thereby resulting in desired modifications of the cell and not just the membrane. Since it is an in vivo system, the cell remains functional.

For example, an amphipathic molecule type D having a functional group in the hydrophilic region, and in particular a chelate group as the functional group, can be added to the mixture according to the invention having molecule types A, B and, optionally, C. The mixture then comprises, for example, the molecule types A, B, D and, optionally, C. The functional group is located on the surface of the cell membrane during fusion of the mixture or the liposome with the cell membrane of the cell.

This approach permits the surface of the cell membrane to be modified and functionalized in a specific manner. To this end, the functional group extends outwardly from the surface of the cell and can be modified in a specific manner by further chemical groups. In a particularly advantageous embodiment of the invention, this can lead to successful cancer therapy.

For this purpose, a mixture according to the invention therefore comprises, for example, 1,2-dioleoyl-3-trimethylammonium-propane as molecule type A and N-(4,4-difluoro-5,7-dimethyl-4-bora-3a,4a-diaza-s-indacene-3-propionyl)-1,2-dihexadecanoyl-sn-glycero-3-phosphoethanolamine as molecule type B and triethylammonium salt/1,2-dioleoyl-sn-glycero-3-phosphoethanolamine as molecule type C and 1,2-dioleoyl-sn-glycero-3-[(N-(5-amino-1-carboxypentyl)iminodiacetic acid)succinyl](nickel salt) as molecule type D, e.g. at a mixing ratio of 1:0.1:1:0.002 wt/wt.

The aforementioned further cell modification then takes place by adding tumor necrosis factor (TNF)α bound to a 6× histidine repeat. This leads to successful cancer therapy, as indicated in method 3 (see below).

An amphipathic molecule type E can be added to the mixture and can be located in the membrane of the cell during fusion, in particular bacteriorhodopsin or glycophorin A or integrin as molecule type E.

Furthermore, a non-amphipathic, hydrophilic molecule type F in a buffered solution can be added to a pre-dried mixture and delivered into the lumen of the cell during fusion. Molecule type F comprises, in particular, RNA, DNA, Ca²⁺, peptide, protein or a pharmaceutical such as acetylsalicylic acid.

A non-amphipathic, hydrophobic molecule type G can also be added to the mixture simultaneously and can be located in the membrane of the cell during fusion. In particular, cholesterol, vitamin A, D, E and K or a pharmaceutical such as cortisone are hereby included.

All molecule types A, B, C, D, and optionally G, can be dissolved and homogenized in an organic solvent. They can then be dried, and dissolved in an aqueous buffer and stored. The aforementioned molecule types E and/F can be added to the solution in the aqueous buffer. Depending on the nature of the molecule type, liposomes of molecule types A, B, C, D, E, F, G can be advantageously formed.

The following molecules can be used to produce a fusion mixture for modifying a cell membrane by way of fusion with the membrane. The following list is not final or limiting.

### Exemplary Embodiments of Molecule Type A:

The criteria for molecule A are that (a) the molecule comprises a hydrophilic region having one or more positive charges, and therefore the total charge of the hydrophilic part of the molecule is positive. The task of this molecule is to bring the fusion mixture into the vicinity of the negatively charged cell membrane by way of electrostatic forces. (b) It also has a hydrophobic region, preferably a C₁₀-C₃₀ component with or without double bonds. Double bonds advantageously cause the membrane of the resulting liposome to become elastic, thereby simplifying fusion of the liposome with the cell membrane. (c) The portion of molecule type A in the mixture according to the invention can be up to 99 wt/wt %.

Suitable molecules are, for example, 1,2-dioleoyl-3-trimethylammonium-propane (DOTAP), N-(2,3-dioleyloxypropyl)-N,N,N-trimethylammonium chloride (DOTMA), dimethyldioctadecylammoniumbromide (DDAB), or (1-[2-(oleoyloxy)ethyl]-2-oleyl-3-(2-hydroxyethyl)imidazolinium chloride (DOTIM). A first example is DOTAP (1,2-dioleoyl-3-trimethylammonium-propane (chloride salt)).

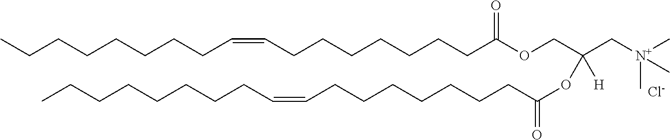

### Exemplary Embodiments of Molecule Type B:

The criteria for amphipathic molecule type B as a fusion-inducing molecule according to the invention are as follows. (a) It must have a hydrophilic region with or, preferably, without a charge. (b) and it must have a hydrophobic region, preferably a C₁₀-C₃₀ component with or without double bonds. Refer to type A for the function of the double bonds: (c) The molecule comprises a group (R) either in the hydrophobic region and/or in the hydrophilic region, which has a delocalized electron system. What is meant is a cyclic structural motif made of conjugated double bonds and/or free electron pairs and/or unoccupied p orbitals.

Particularly preferably, there is a large electronegativity difference between covalently bound adjacent atoms in the R group. Particularly advantageously, it can be at least 0.4 (←_{χ}≧0.4). This serves to increase polarizability and influences fusion in a positive manner. (d) The portion of fusion-inducing molecule type B in the mixture can be adjusted to up to 40 wt/wt %.

The table below lists a few relevant R groups for the molecules of type B.

TABLE 1
Summary of properties of fusion-inducing and non-fusion-inducing R groups bound to
amphipathic molecules B (e.g.: phospholipids, such as DHPE or DOPE, for example).

| Fusion- | | Number of | | | |
|---|---|---|---|---|---|
| inducing group | Number | delocalized | | Covalently bound to | Fusion |
| (R) | of rings | electrons | Greatest Δχ | lipid | quality ^{a} |
| BODIPY | 3 | 10 | F—B(4 − 2 = 2) | chain of C₁₂HPC | 1 |
| BODIPY | 3 | 10 | F—B(4 − 2 = 2) | head of DHPE | 1 |
| DiO per se | 2 × 2 | 2 × 8 | O—C(3.4 − 2.5 = 0.9) | — | 1 |
| LR | 3 + 1 | 12 + 6 | O—C(3.4 − 2.5 = 0.9) | head at DHPE/DOPE | 1 |
| Texas-Red | 7 + 1 | 14 + 6 | O—C(3.4 − 2.5 = 0.9) | head at DHPE | 1 |
| NBD | 2 | 8 | N—C(3 − 2.5 = 0.5) | head at DOPE | 2 |
| fluorescein | 3 + 1 | 12 + 6 | O—C(3.4 − 2.5 = 0.9) | head at DHPE | 3 |
| pyrene | 4 | 16 | C—H(2.5 − 2.1 = 0.4) | chain at C₁₀HPC | 5 |
| pyrene | 4 | 16 | C—H(2.5 − 2.1 = 0.4) | head at DOPE | 5(toxic) |
| capBiotin | 2 | 0 | N—C(3 − 2.5 = 0.5) | head at DOPE | 6 |
| inositol | 1 | 0 | O—O(3.4 − 2.5 = 0.9) | | 6(toxic) |
| PEG(2000) | 0 | 0 | O—O(3.4 − 2.5 = 0.9) | head at DOPE | 6(toxic) |

^{a} 1—very good, 2—good, 3—satisfactory, 4—adequate, 5—barely functional, 6—non-functional

### First Example of Molecule Type B (β-BODIPY-C₁₂-HPC)

2-(4,4-difluoro-5-methyl-4-bora-3a,4a-diaza-s-indacene-3-dodecanoyl) is β-BODIPY and is the R group. This R group is bound to 1-hexadecanoyl-2-dodecanoyl-sn-glycero-3-phosphocholine (C₁₂—HPC). The two together result in the following structural formula:

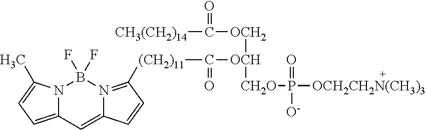

### Second Example of Molecule Type B (BODIPY FL DHPE)

N-(4,4-difluoro-5,7-dimethyl-4-bora-3a,4a-diaza-s-indacene-3-propionyl) is BODIPY-FL. This R group is bound to DHPE (1,2-dihexadecanoyl-sn-glycero-3-phosphoethanolamine (triethylammonium salt)). The two together result in the following structural formula:

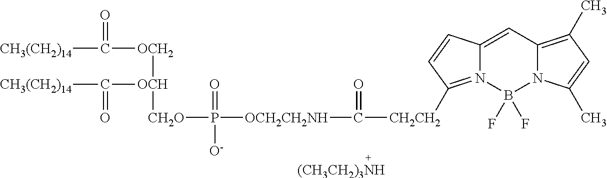

### Third Example of Molecule Type B (‘DiO’)

DiOC₁₈(3)3,3′-dioctadecyloxacarbocyanine perchlorate. This molecule is not a lipid-bound dye as with the first two B molecules, but is an amphipathic molecule per se having the structural formula:

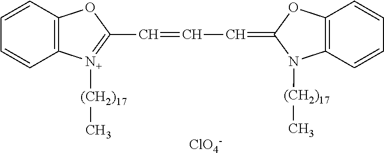

### Fourth Example of Molecule Type B (LR-DOPE)

Lissamine rhodamine B sulfonyl is the R group. It is bound to 1,2-dioleoyl-sn-glycero-3-phosphoethanolamine (DOPE) as the amphipathic molecule. The two together result in the structural formula:

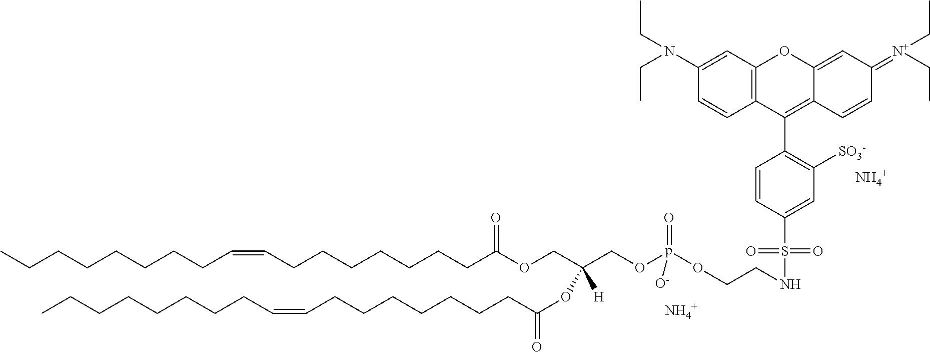

### Fifth Exemplary Embodiment of Molecule Type B (Texas Red® DHPE)

Texas Red is (5-(chlorosulfonyl)-2-(1H,2H,3H,5H,6H,7H,11H,12H,13H,15H,16H,17H-pyrido[3,2,1-ij]quinolizino[1′,9′6,7,8]chromeno[2,3-f]quinolin-4-ium-9-yl)benzene sulphonate, i.e. the R group. It is bound to 1,2-dihexadecanoyl-sn-glycero-3-phosphoethanolamine and, together, results in the structural formula:

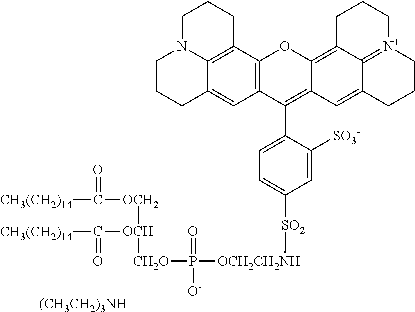

### Sixth Example of Molecule Type B (NBD-DOPE)

NBD is (7-nitro-2-1,3-benzoxadiazol-4-yl), which is to say the R group. It is bound to 1,2-dioleoyl-sn-glycero-3-phosphoethanolamine having the structural formula:

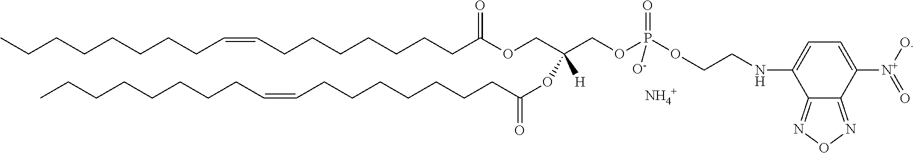

### Seventh Example of Molecule Type B (Fluorescein-DOPE)

Carboxyfluorescein is the R group and is bound to 1,2-dioleoyl-sn-glycero-3-phosphoethanolamine having the structural formula:

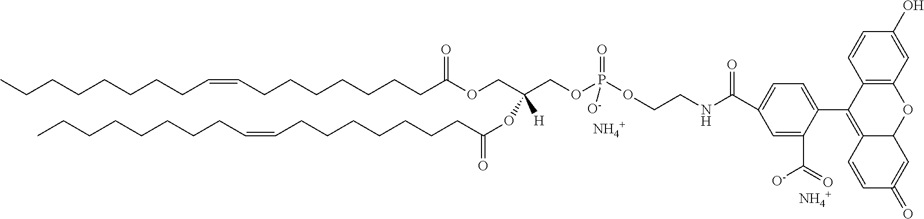

### Eighth Example of Molecule Type B (Pyrene-DOPE)

1-Pyrenesulfonyl is the R group and is bound to 1,2-dioleoyl-sn-glycero-3-phosphoethanolamine having the structural formula:

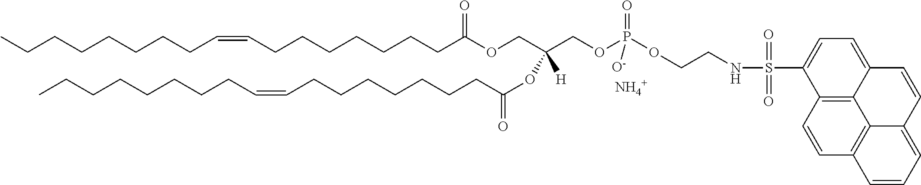

Ninth Example of Molecule Type B (β-py-C₁₀-HPC)

Pyrenedecanoyl is the R group and is bound to 1-hexadecanoyl-2-sn-glycero-3-phosphocholine having the structural formula:

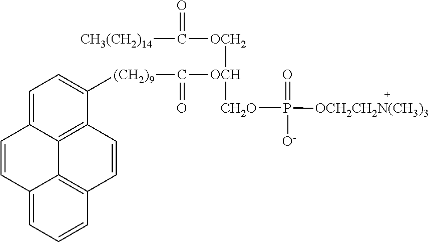

According to the invention, all of the R groups have a delocalized electron structure. Due to the delocalization, the R group is often present as a dye in molecule type B.

It is feasible for the R group itself to be formed by a hydrophobic part of an amphipathic molecule and by delocalized double bonds.

Table 1 also shows that the fusion quality improves with greater differences between directly adjacent atoms in terms of the electronegativity thereof.

The following molecules, such as capBio-DOPE, do not, however, function:

cap Biotinyl would be the R group bound to 1,2-dioleoyl-sn-glycero-3-phosphoethanolamine having the structural formula:

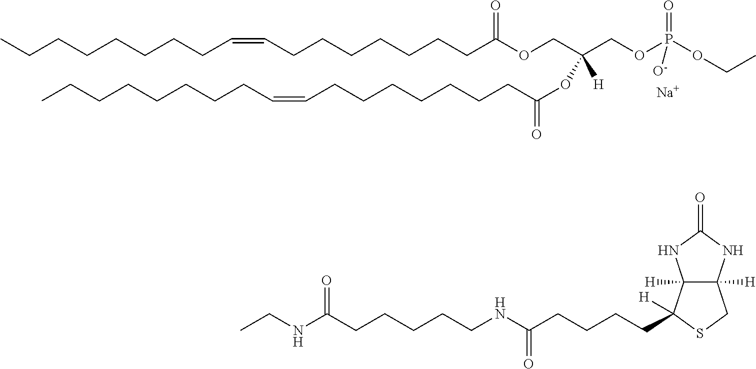

Likewise non-functional is PI: L-α-phosphatidylinositol (sodium salt)

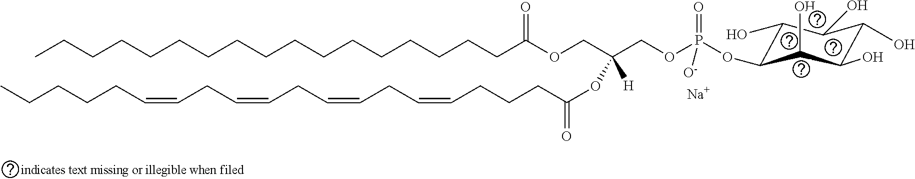

and

Methoxy(polyethylene glycol)-2000 would be the R group bound to 1,2-dioleoyl-sn-glycero-3-phosphoethanolamine (PEG2000-DOPE) having the structural formula:

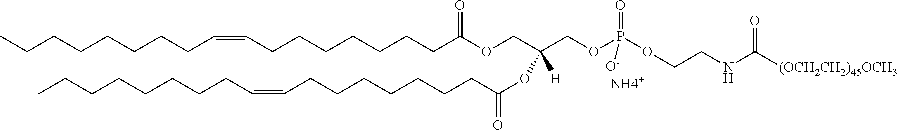

Delocalization of the electron structure is not present in the last three aforementioned molecules. This shows that the mixture according to the invention must have this.

As described, it is feasible to combine the positive properties of molecule type A with respect to the positive charge and those of molecule type B with respect to the delocalized electron system, or with respect to the differences in electronegativity, in one chemically synthesized molecule. This molecule, which was not previously known to the inventors, should therefore likewise be a subject of the invention.

Optionally, and for the further advantageous embodiment of the mixture, the following further molecules for producing a fusion mixture or for modifying a cell membrane are possible.

### Exemplary Embodiments of Molecule Type C as a Helper Molecule

The criteria for the helper molecule/carrier molecule C are: (a) The molecule must have a hydrophilic region and (b) a hydrophobic region (in particular C₁₀-C₃₀) with or without double bonds. Refer to type A and/or B for the function of the double bonds. (c) Both regions should have be neutral in order to neutralize the great charge density and the repellent forces between positively charged molecules of molecule type A. This effect stabilizes the system. (d) The portion of the carrier molecule can be adjusted up to a maximum of 70 wt %/wt.

Suitable molecules are, for example, phosphatidylethanolamine, phosphatidylcholine (1,2-dioleoyl-sn-glycero-3-phosphoethanolamine, 1,2-dipalmitoyl-sn-glycero-3-phosphoethanolamine, 1,2-dimyristoyl-sn-glycero-3-phosphoethanolamine, 1,2-dielaidoyl-sn-glycero-3-phosphoethanolamine, 1,2-diphytanol-sn-glycero-3-phosphoethanolamine, 1,2-dilinoleoyl-sn-glycero-3-phosphoethanolamine or 1,2-dioleoyl-sn-glycero-3-phosphatidylcholine).

The first example is 1,2-dioleoyl-sn-glycero-3-phosphoethanolamine (DOPE).

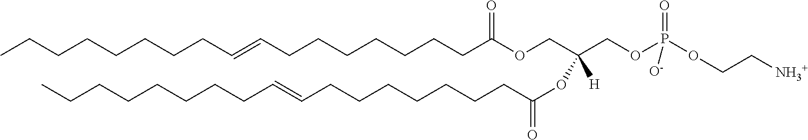

Advantageously, it is possible to form molecule types A, B and C as lipids or as polymers. In the case of amphipathic lipids, liposomes are formed when producing the ready-for-use mixture.

The mixing ratio by weight (wt/wt) between molecule types A, B and C is advantageously 1:0.05-0.2:1.

Molecule Type D (Amphipathic with Functional Group)

The criteria for amphipathic molecule type D are: (a) The molecule must have a hydrophilic region and (b) a hydrophobic region (in particular with C₁₀-C₃₀) with or without double bonds. Refer to types A, B or C for the function of the double bonds. (c) The molecule must have a functional group in the hydrophilic region which permits further chemical bonds on the cell surface after fusion.

Suitable molecules are, for example, long-chained fatty acids and alcohols, chelate complex-modified lipids such as 1,2-dioleoyl-sn-glycero-3-[(5-amino-1-carboxypentyl)iminodiacetic acid)succinyl](nickel salt), biotin-modified lipids (1,2-dioleoyl-sn-glycero-3-phosphoethanolamine-N-cap-Biotin), or PEG-modified lipids (1,2-dioleoyl-sn-glycero-3-phosphoethanolamine-N-[methoxy(polyethylene glycol)-2000](ammonium salt) or pharmaceuticals.

A first exemplary embodiment thereof is (iminodiacetic acid)succinyl (nickel salt) as the functional group, in the form of a chelate group in this case, bound to 1,2-dioleoyl-sn-glycero-5-amino-1-carboxypentyl. This results in the structural formula:

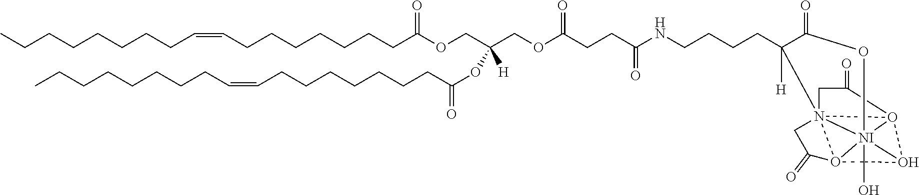

### Molecule Type E (Amphipathic):

The criteria for molecule type E are, furthermore: (a) They are amphipathic. (b) They can be membrane proteins, peptides, surface receptors, such as bacteriorhodopsin, integrin, glycophorin A and many others. (c) Molecule type E, similar to the other components A, B, C, D, can be synthesized artificially or obtained from cells. (d) Modification of these molecules, such as fluorescence or radioactive marking, can be present. (e) Molecule type E can be added to the mixture at a ratio of up to 20 wt %/wt.

The amphipathic molecule type E is incorporated into the liposome membrane when lipids are used as type A-D. Non-amphipathic molecule types E are incorporated bound to the liposome membrane when lipids are used as type A-D.

### Molecule Type F (Water Soluble or Hydrophilic):

The criteria for molecule type F are: (a) The molecules should be water soluble. Examples are: ions, peptides, proteins, pharmaceuticals, DNA or RNA.

When lipids are used as molecule types, molecule type F is incorporated into the lumen of the liposome.

### Molecule Type G (Non-Water Soluble or Hydrophobic):

The criteria for molecule type G are: (a) The molecules are hydrophobic and are readily soluble in organic solvent. Examples are: cholesterol, vitamin B. They are mixed together with molecule types A, B, C, and D in an organic solvent.

### The Fusion Procedure

The components of the mixture according to the invention with molecule types A and B are taken up in the desired ratio by weight together with molecules C, optionally D and optionally G, which are further optional molecules and are mixed therewith, simultaneously in an organic solvent, preferably in chloroform, methanol, ethanol, propanol, hexane, heptane or a mixture thereof. After homogenization in the organic solvent, the organic solvent should be removed.

The dried mixture is taken up in an aqueous buffer solution, preferably having a pH value of 7, and is homogenized once more. Advantageously a liposome forms, which comprises the molecule types that were added. Molecules of molecule types E and F can likewise be added.

The mixtures are stable in this form, when stored at 4° C. for at least one month, for example. These steps serve as preparation for the method according to the invention.

According to the actual method, the fusion mixture is diluted with a cell medium, e.g. 1:100 (mixture:cell medium v/v), homogenized once more, and added to the cells.

Fusion is preferably brought about in vivo by adding the fusion mixture according to the invention to living cells. To this end it is sufficient for the cells to be brought in contact with the mixture for approximately 1 to 60 minutes, and in the case of tissue samples, for 60 to 120 minutes, for example. A cell washing step should then be incorporated.

The method is advantageously characterized by a particularly high fusion efficiency of at least 50%, preferably more than 70%, and in particular more than 90%.

Particularly advantageously, the membrane fusion is not limited to individual cell types, but rather is a nearly universal mechanism, as shown in Table 2. Even dense tissue samples can be successfully treated by way of an extended incubation period. The method can therefore be used in a highly versatile manner, to particular advantage.

In the fusion of the mixture according to the invention with a cell membrane, the vesicular volumes are incorporated into the cell in the form of molecule type F, which can be used advantageously to incorporate soluble molecules, ions, proteins or DNA into the cytoplasma of the cells. Molecule type F is therefore not a component of the mixture of amphipathic molecule types per se.

Molecule types D, E or G, which are biologically or pharmaceutically relevant, can be incorporated into intact cell membranes during the fusion procedure. The reactive groups of molecule type D incorporated in the cell membrane can be used for coupling reactions, for example. Additional membrane-specific molecules of type E, such as peptides or proteins, can be incorporated into the cell membrane.

The mixture according to the invention and the method according to the invention make it possible to perform analyses in a multiplicity of fields of basic research, such as diffusion analysis of molecules incorporated into intact cell membranes, the characterization of lipid micro-domains and nano-domains, which are also known as “lipid rafts” in the literature, or in investigating regulation and transport pathways of membrane molecules in the cells.

The system can also be used, for example, for the medically highly relevant, specific marking of certain cell types, e.g. to mark cancer cells, to support wound closure by inducing cell migration, to form cell-cell contacts, of cell projections or proliferation induction or cell-cell fusion. Certain amphipathic mixtures, according to the invention, of molecule types A, B, (C), D with TNFα can therefore be used as drugs and simultaneously for the specific marking and handling of cancer cells.

Table 2 provides an overview of a few methods for cell membrane modification.

TABLE 2
Summary of cell modification experiments using the fusion mixture.

| | Fusion mixture | | | | | | |
|---|---|---|---|---|---|---|---|
| | Type | Type | Type | Type | | | |
| No | C | A | B^{a} | D-G | Method | Cell type | Application |
| 1 | DOPE | DOTAP | LR-DOPE | — | 1 | HEK | Cell |
| | | | | | | | membrane |
| | | | | | | | staining |
| 2 | DOPE | DOTAP | Texas-Red- | — | 1 | HEK | Cell |
| | | | DHPE | | | | membrane |
| | | | | | | | staining |
| 3 | DOPE | DOTAP | NBD-DOPE | — | 1 | HEK | Cell |
| | | | | | | | membrane |
| | | | | | | | staining |
| 4 | DOPE | DOTAP | BODIP Y | — | 1 | HEK | Cell |
| | | | FL DHPE | | | | membrane |
| | | | | | | | staining |
| 5 | DOPE | DOTAP | B-BODIP Y | — | 1 | HEK | Cell |
| | | | C₁₂-HPC | | | | membrane |
| | | | | | | | staining |
| 6 | DOPE | DOTAP | fluorescein- | — | 1 | HEK | Cell |
| | | | DHPE | | | | membrane |
| | | | | | | | staining |
| 7 | DOPE | DOTAP | DiO | — | 1 | HEK | Cell |
| | | | | | | | membrane |
| | | | | | | | staining |
| 8 | DOPE | DOTAP | Pyrene- | — | 1 | HEK | Cell |
| | | | DOPE | | | | membrane |
| | | | | | | | staining |
| 9 | DOPE | DOTAP | β-Pyrene | — | 1 | HEK | Cell |
| | | | C₁₀-HPC | | | | membrane |
| | | | | | | | staining |
| 10 | DOPE | DOTAP | β-BODIP | PI^{d} | 2 | HEK | PI function |
| | | | Y C₁₂-HPC | | | | checks |
| 11 | DOPE | DOTAP | LR-DHPE | CaCl₂ | 3 | HEK | Drug delivery |
| 12 | DOPE | DOTAP | BODIP | BR-TRITC^{b} | 4 | HEK | Prot. function |
| | | | Y FL | | | | checks |
| | | | DHPE | | | | |
| 13 | DOPE | DOTAP | BODIP | GPA-TRITC^{b} | 4 | HEK | Prot. function |
| | | | Y FL | | | | checks |
| | | | DHPE | | | | |
| 14 | DOPE | DOTAP | β-BODIP | DOGS- | 2 | HEK | His-tag binding |
| | | | Y C₁₂-HPC | NTA^{c} | | | |
| 15 | DOPE | DOTAP | LR-DHPE | DOGS- | 2 | HEK | Activation of |
| | | | | NTA^{c} | | | macrophages |
| | | | | | | | by way of TNF |
| | | | | | | | binding |
| 16 | DOPE | DOTAP | BODIP | DOGS- | 2 | HEK | Activation of |
| | | | Y FL | NTA^{c} | | | macrophages |
| | | | DHPE | | | | by way of TNF |
| | | | | | | | binding |
| 17 | DOPE | DOTAP | LR-DOPE | — | 1 | Myofibroblasts | Cell |
| | | | | | | | membrane |
| | | | | | | | staining |
| 18 | DOPE | DOTAP | Texas-Red- | — | 1 | Myofibroblasts | Cell |
| | | | DHPE | | | | membrane |
| | | | | | | | staining |
| 19 | DOPE | DOTAP | fluorescein- | — | 1 | Myofibroblasts | Cell |
| | | | DHPE | | | | membrane |
| | | | | | | | staining |
| 20 | DOPE | DOTAP | β-BODIP | — | 1 | Myofibroblasts | Cell |
| | | | Y C₁₂-HPC | | | | membrane |
| | | | | | | | staining |
| 21 | DOPE | DOTAP | β-BODIP | DOGS- | 2 | Myofibroblasts | His-tag binding |
| | | | Y C₁₂-HPC | NTA^{c} | | | |
| 22 | DOPE | DOTAP | β-BODIP | DOGS- | 2 | Myofibroblasts | Activation of |
| | | | Y C₁₂-HPC | NTA^{c} | | | macrophages |
| | | | | | | | by way of TNF |
| | | | | | | | binding |
| 23 | DOPE | DOTAP | Texas-Red- | capBioDOPE^{c} | 2 | Myofibroblasts | Avidin binding |
| | | | DHPE | | | | to the cell |
| | | | | | | | surface |
| 24 | DOPE | DOTAP | Texas-Red- | BFL-SM^{c} | 2 | Myofibroblasts | Marking of SM |
| | | | DHPE | | | | in the plasma |
| | | | | | | | membrane |
| 25 | DOPE | DOTAP | β-BODIP | BR-TRITC^{b} | 4 | Myofibroblasts | Prot. function |
| | | | Y C₁₂-HPC | | | | checks |
| 26 | DOPE | DOTAP | β-BODIP | GPA-TRITC^{b} | 4 | Myofibroblasts | Prot. function |
| | | | Y C₁₂-HPC | | | | checks |
| 27 | DOPE | DOTAP | LR-DOPE | — | 1 | keratinocytes | Cell |
| | | | | | | | membrane |
| | | | | | | | staining |
| 28 | DOPE | DOTAP | β-BODIP | — | 1 | keratinocytes | Cell |
| | | | Y C₁₂-HPC | | | | membrane |
| | | | | | | | staining |
| 29 | DOPE | DOTAP | LR-DOPE | DOGS- | 2 | keratinocytes | His-tag binding |
| | | | | NTA^{c} | | | |
| 30 | DOPE | DOTAP | β-BODIP | DOGS- | 2 | keratinocytes | His-tag binding |
| | | | Y C₁₂-HPC | NTA^{c} | | | |
| 31 | DOPE | DOTAP | β-BODIP | — | 1 | R37 | Cell |
| | | | Y C₁₂-HPC | | | | membrane |
| | | | | | | | staining |
| 32 | DOPE | DOTAP | β-BODIP | DOGS- | 2f | R37 | His-tag binding |
| | | | Y C₁₂-HPC | NTA^{C} | | | |
| 33 | DOPE | DOTAP | LR-DHPE | — | 1 | BSMC | Cell |
| | | | | | | | membrane |
| | | | | | | | staining |
| 34 | DOPE | DOTAP | β-BODIP | — | 1 | BSMC | Cell |
| | | | Y C₁₂-HPC | | | | membrane |
| | | | | | | | staining |
| 35 | DOPE | DOTAP | β-BODIP | BR-TRITC^{b} | 4 | BSMC | Prot. function |
| | | | Y C₁₂-HPC | | | | checks |
| 36 | DOPE | DOTAP | LR-DHPE | — | 1 | ESMC | Cell |
| | | | | | | | membrane |
| | | | | | | | staining |
| 37 | DOPE | DOTAP | β-BODIP | — | 1 | ESMC | Cell |
| | | | Y C₁₂-HPC | | | | membrane |
| | | | | | | | staining |
| 38 | DOPE | DOTAP | β-BODIP | BR-TRITC^{b} | 4 | ESMC | Prot. function |
| | | | Y C₁₂-HPC | | | | checks |
| 39 | DOPE | DOTAP | LR-DHPE | — | 1 | h. fibroblasts | Cell |
| | | | | | | | membrane |
| | | | | | | | staining |
| 40 | DOPE | DOTAP | β-BODIP | — | 1 | h. fibroblasts | Cell |
| | | | Y C₁₂-HPC | | | | membrane |
| | | | | | | | staining |
| 41 | DOPE | DOTAP | LR-DHPE | — | 1 | Macrophages | Cell |
| | | | | | | | membrane |
| | | | | | | | staining |
| 42 | DOPE | DOTAP | β-BODIP | — | 1 | Macrophages | Cell |
| | | | Y C₁₂-HPC | | | | membrane |
| | | | | | | | staining |
| 43 | DOPE | DOTAP | β-BODIP | BR-TRITC^{b} | 4 | Macrophages | Prot. function |
| | | | Y C₁₂-HPC | | | | checks |
| 44 | DOPE | DOTAP | LR-DHPE | — | 1 | Neurons | Cell |
| | | | | | | | membrane |
| | | | | | | | staining |
| 45 | DOPE | DOTAP | β-BODIP | — | 1 | Neurons | Cell |
| | | | Y C₁₂-HPC | | | | membrane |
| | | | | | | | staining |
| 46 | DOPE | DOTAP | β-BODIP | BR-TRITC^{b} | 4 | Neurons | Prot. function |
| | | | Y C₁₂-HPC | | | | checks |
| 47 | DOPE | DOTAP | BODIP | | 1 | Pericardium | Cell |
| | | | Y FL- | | | | membrane |
| | | | DHPE | | | | staining |

^{a}Molecule type C/molecule type A molecule type B = 1/1/0.1 wt/wt in every experiment performed.
^{b}Refer to method 2, below, for the exact composition.
^{c}Refer to method 3, below, for the exact composition.
^{d}DOPE/DOTAP/β-BODIPY C₁₂-HPC/PI = 1/1/0.1/0.1

### Abbreviations Used in Table 2:

Method 1: cell membrane staining; method 2: cell surface modification; method 3: cell lumen modification; method 4: protein reconstruction in the cell membrane

For molecule type C: DOPE=1,2-dioleoyl-sn-glycero-3-phosphoethanolamine

For molecule type A: DOTAP=1,2-dioleoyl-sn-glycero-(trimethylammonium-propane (chloride salt)

### For Molecule Type B:

β-BODIPY C₁₂—HPC: 2-(4,4-difluoro-5-methyl-4-bora-3a,4a-diaza-s-indacene-3-dodecanoyl)-1-hexadecanoyl-sn-glycero-3-phosphocholine

β-pyrene C₁₀-HPC: 1-hexadecanoyl-2-(1-pyrenedecanoyl)-sn-glycero-3-phosphocholine

BODIPY FL DHPE: N-(4,4-difluoro-5,7-dimethyl-4-bora-3a,4a-diaza-s-indacene-3-propionyl)-1,2-dihexadecanoyl-sn-glycero-3-phosphoethanolamine, (triethylammonium salt)

DiO: DiOC₁₈(3)3,3′-dioctadecyloxacarbocyanine perchlorate

Fluorescein DHPE: 1,2-dioleoyl-sn-glycero-3-phosphoethanolamine-N-(carboxyfluorescein) (ammonium salt)

LR-DHPE: Lissamine rhodamine B 1,2-dihexadecanoyl-sn-glycero-3-phosphoethanolamine, (triethylammonium salt)

LR-DOPE: Lissamine rhodamine B 1,2-diolenoyl-sn-glycero-3-phosphoethanolamine, (triethylammonium salt)

NBD-DOPE: N-(7-nitrobenz-2-oxa-1,3-diazol-4-yl)-1,2-diolenoyl-sn-glycero-3-phosphoethanolamine (triethylammonium salt)

Pyrene-DOPE: 1,2-dioleoyl-sn-glycero-3-phosphoethanolamine-N-(1-pyrenesulfonyl) (ammonium salt)

Texas Red DHPE: Texas Red 1,2-dihexadecanoyl-sn-glycero-3-phosphoethanolamine (triethylammonium salt)

### For Molecule Types D-G:

BR-TRITC: Bacteriorhodopsin from Halobacterium salinarum labelled with tetramethylrhodamine isothiocyanate mixed isomers as molecule type E.

DOGS-NTA: 1,2-dioleoyl-sn-glycero-3-[(N-(5-amino-1-carboxypentyl)iminodiacetic acid)succinyl](nickel salt) as molecule type D.

capBioDOPE: 1,2-dioleoyl-sn-glycero-3-phosphoethanolamine-N-cap-biotin as molecule type D

B FL-SM: N-(4,4-difluoro-5,7-dimethyl-4-bora-3a,4a-diaza-s-indacene-3-dodecanoyl)sphingosyl phosphocholine as molecule type D

GPA-TRITC: Glycophorin A (MNS blood group) labelled with tetramethylrhodamine isothiocyanate mixed isomers as molecule type E.

PI: L-α-phosphatidyl-inositol (sodium salt) as molecule type E.

CaCl₂×H₂O as molecule type F.

### Cell Types and Cell Lines:

BSMC: Bronchial smooth muscle cells

ESMC: Embryonal smooth muscle cells

HEK: Human Embryonic Kidney, HEK293

h. fibroblasts: human fibroblasts

Keratinocytes: human keratinocytes

Macrophages: human macrophage

Myofibroblasts: rat cardiac fibroblasts

Neurons: rat embryonal cortical neurons

R37: breast cancer cell line

Heart sac: rat embryonic heart sac

### Other Matters in Table 2:

TNF: TNFα-His-Tag: tumor necrosis factor linked with 6× histidine repeat The invention will be explained hereafter in greater detail with reference to exemplary embodiments and the attached figures, although this is not intended to limit the invention.

### They Show:

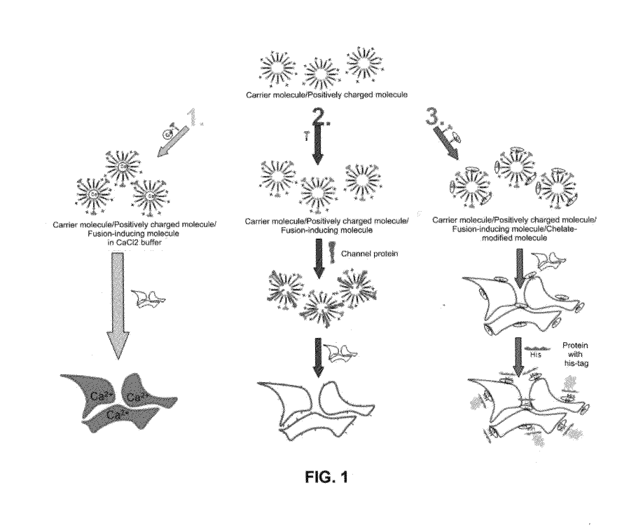

FIG. 1: Schematic depiction of three possible uses of the fusion reagent.

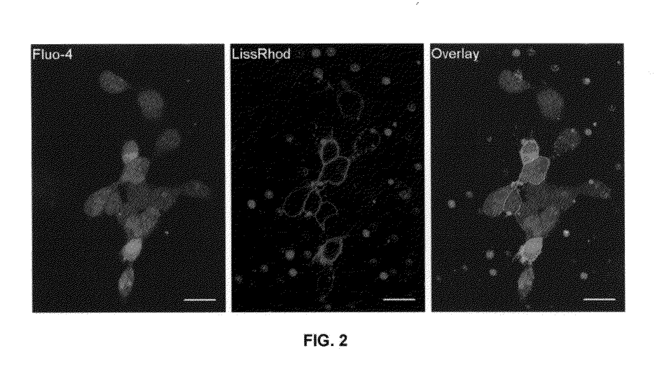

FIG. 2: Corresponds to line 11 in Table 2. Microscopic photographs of HEK293 cells pre-incubated in Fluo-4 AM after addition of the fusion mixture (DOPE (molecule type C)/DOTAP (molecule type A)/LR-DHPE (molecule type B)=1/1/0.1 wt/wt) filled with 1 mM CaCl₂ (molecule type F) solution. The red-marked lipid mixture (molecule types A-C) (LissRhod channel) were incorporated into the cell membrane, while the green Ca²⁺ indicator (Fluo-4 channel) indicates an elevated Ca²⁺ concentration (molecule type F) in the cell membrane. (Scale=20 μm).

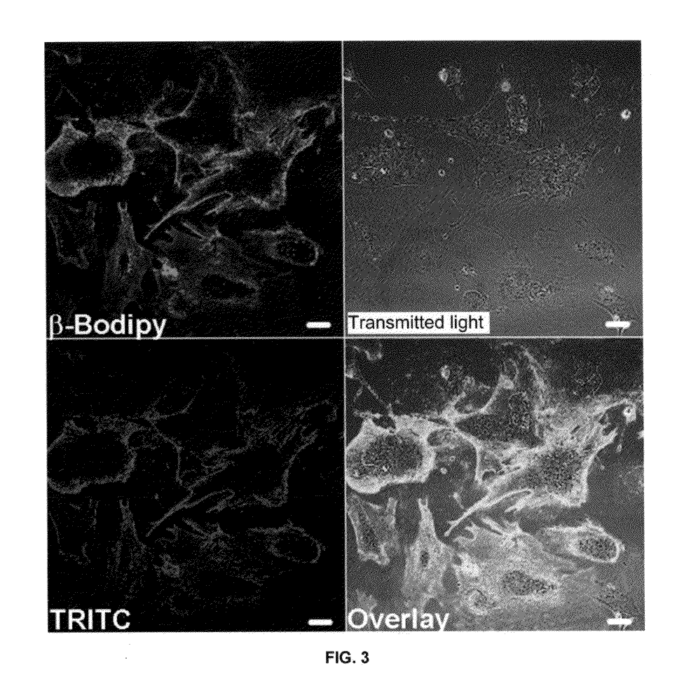

FIG. 3: Corresponds to line 25 in Table 2. Microscopic photographs of fibroblasts 10 minutes after addition of the fusion mixture (DOPE (molecule type C)/DOTAP (molecule type A)/β-Bodipy-C₁₂HPC (molecule type B)/BR-TRITC (molecule type E)=1/1/0.1/0.0005 wt/wt). The green-marked lipid mixture (molecule types A-C) (β-Bodipy channel) and the red-marked channel protein (molecule type E) (TRITC channel) were incorporated into the cell membrane. (Scale=20 μm)

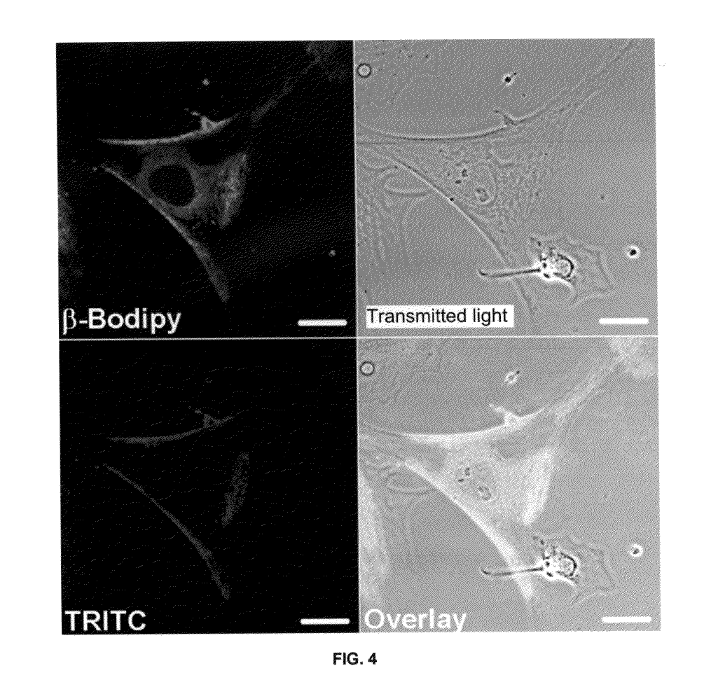

FIG. 4: Corresponds to line 25 in Table 2. Microscopic photographs of fibroblasts 4 hours after the fusion mixture was washed away (DOPE (molecule type C)/DOTAP (molecule type A)β-Bodipy-C₁₂HPC (molecule type B)/BR-TRITC (molecule type E)=1/1/0.1/0.0005 wt/wt). The green-marked lipid mixture (molecule types A-C) (β-Bodipy channel) and the red-marked channel protein (molecule type E) (TRITC channel) were incorporated into the cell membrane. The separation of the two dyes as a function of time indicates a different transport pathway of the membrane lipids and proteins from the cell membrane into the cell interior. (Scale=20 μm).

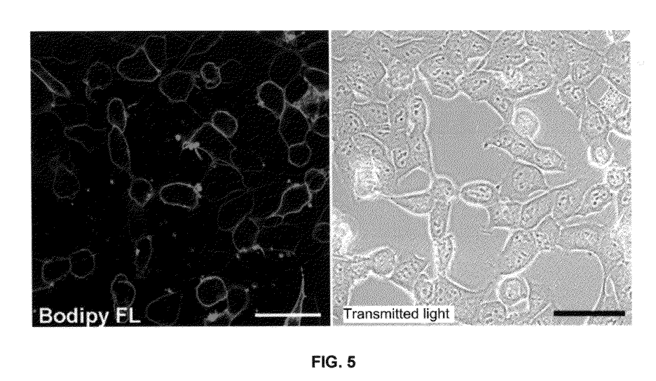

FIG. 5: Corresponds to line 16 in Table 2. Microscopic photographs of HEK293 cells 10 minutes after addition of the fusion mixture (DOPE (molecule type C)/DOTAP (molecule type A)/Bodipy FL-DHPE (molecule type B)/DOGS-NTA (molecule type D)=1/1/0.1/0.002 wt/wt). (Scale=50 μm).

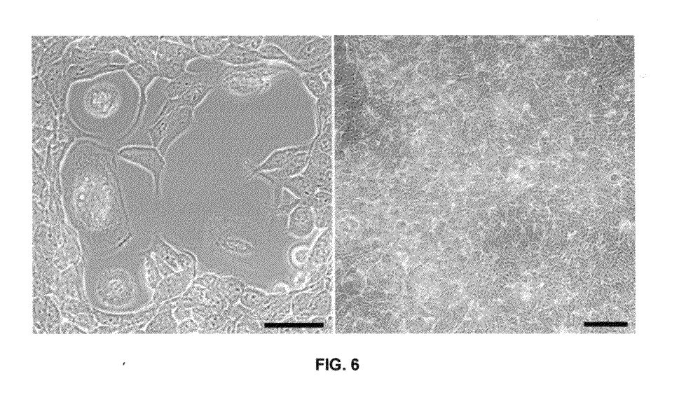

FIG. 6: Corresponds to line 16 in Table 2. Microscopic photographs of TNFα-His-Tag treated HEK293 cells with incorporated DOGS-NTA (molecule type D) in the cell membrane and macrophages and B: Microscopic photograph of TNFα-His-Tag treated HEK293 cells without DOGS-NTA (molecule type D) in the cell membrane and macrophages. (Scale=50 μm).

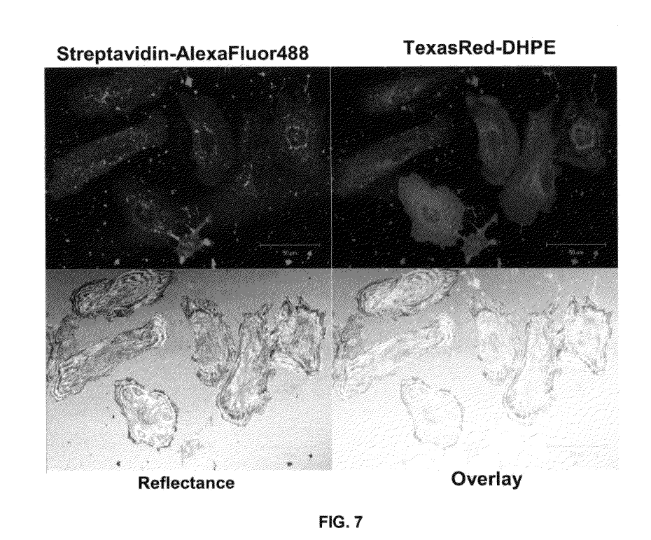

FIG. 7: Corresponds to line 23 in Table 2. Microscopic photographs of fibroblasts after the fusion mixture was washed away (DOPE (molecule type C)/DOTAP (molecule type A)/TexasRedDHPCC (molecule type B)/capBioDOPE (molecule type D)=1/1/0.1/0.01 wt/wt). The red-marked lipid mixture (molecule type A-C) (TexasRed channel), and the green-marked surface protein (AlexaFluor488 channel) indicates unambiguous co-localization of phospholipid and proteins bound to the cell surface. (Scale=50 μm).

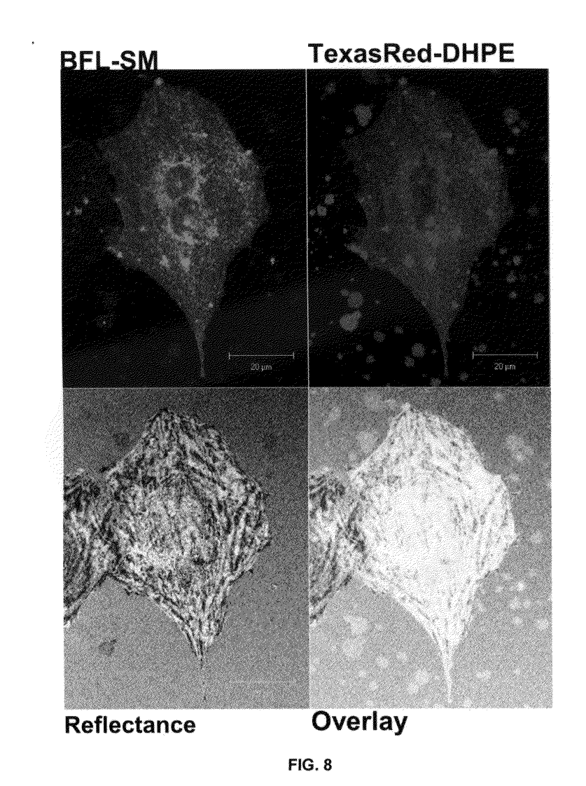

FIG. 8: Corresponds to line 24 in Table 2. Microscopic photographs of fibroblasts after the fusion mixture was washed away (DOPE (molecule type C)/DOTAP (molecule type A)/TexasRedDHPC (molecule type B)/B FL-SM (molecule type D)=0/1/0.1/0.01 wt/wt). The photographs show that the incorporation of the phospholipids (TexasRed channel) and the sphingolipids (BFL channel) was successful. (Scale=20 μm)

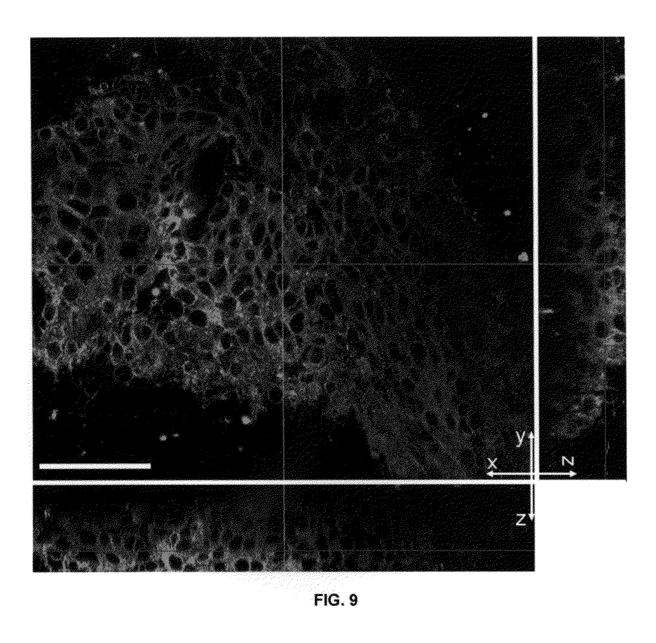

FIG. 9: Corresponds to line 47 in Table 2. Microscopic photograph of primary heart sac tissue (pericardium) from embryonal rats (day 19) after one-hour treatment with the fusion mixture (DOPE (molecule type C)/DOTAP (molecule type A)/Bodipy FL-DHPE (molecule type B)=1/1/0.1 wt/wt). The photograph shows clearly that the fusion mixture of molecule types A-C has penetrated the tissue by approximately 3-4 cell layers and has induced membrane fusion. (Scale=70 μm).

FIG. 1 shows three methods of cell membrane modification according to the invention. Each method is described in a further exemplary embodiment.

Method 1 from FIG. 1: Cell membrane modification by mixing types A-C and addition of Ca²⁺ ions as type F into the lumen of cells.

Human Embryonic Kidney (HEK293) (DSMZ Germany) cells were incubated in DMEM medium (Sigma Aldrich, St. Luis, Mo., USA) at a cell density of 30,000 cells per cell culture dish (Ø3.5 cm) in Fluo-4 AM Ca²⁺ indicator (Invitrogen, Eugene, Oreg., USA) in (1 μg/ml) for 30 minutes. After incubation, the cells were washed with DMEM medium and incubated for 30 minutes with a fusion mixture according to the invention.

The fusion mixture according to the invention is composed of a lipid mixture of 1,2-dioleoyl-sn-glycero-3-phosphoethanolamine (DOPE: molecule type C), 1,2-dioleoyl-3-trimethylammonium-propane (DOTAP: molecule type A) (both from Avanti Polar Lipids Inc., Alabama, Ala., USA), Lissamine rhodamine B 1,2-dihexadecanoyl-sn-glycero-3-phosphoethanolamine (LR-DHPE: molecule type B with R group) (Invitrogen) at a ratio by weight of DOPE/DOTAP/LR-DHPE (1/1/0.1 wt/wt).

The lipid components were first mixed homogeneously in chloroform (VWR, Darmstadt, Germany). The solution was then dried in a vacuum for 30 to 60 minutes at room temperature.

The lipid molecules were taken up once more in a buffer solution of 20 mM (2-[4(2-hydroxyethyl)-1-piperazinyl]-ethanesulphonic acid) (HEPES buffer) (VWR) and 1 mM CaCl₂ as molecule type F (VWR) in a final concentration of 2 mg lipid/ml buffer, and homogenized in an ultrasonic bath (80-100 W) for 20 minutes at room temperature. The mixture is therefore in the form of a liposome. This mixture is stable for approximately 1 month under refrigeration at 4° C. and can be reused after re-homogenization.

5 μl of this fusion mixture are diluted 100-fold with DMEM medium and re-homogenized for 5 to 10 minutes using ultrasound before being added to a cell culture dish containing HEK293 cells incubated with Fluo-4 AM Ca²⁺ indicator (Invitrogen).

In the fusion method according to the invention, the cells are incubated for 10 to 30 minutes with the mixture according to the invention at 37° C. and 5% CO₂. The cells were then washed with DMEM medium and the fusion of the reagent with the cell membrane was examined using a fluorescence microscope (LSM 710 from Carl Zeiss Microimaging GmbH, Jena) (FIG. 2).

After successful membrane fusion, the red fluorescent lipid molecules of molecule type B are incorporated into the cell membrane of the HEK293 cells. The cell membranes are therefore easily recognized under a fluorescence microscope (see FIG. 2-LissRhod channel).

Evidence of delivery of the vesicular lumen into the cell interior and, therefore, of molecule type F as well, is provided by the increased green fluorescence intensity of the Fluo-4 AM Ca²⁺ indicator, which was induced by the increased Ca²⁺ concentration in the cell volume after membrane fusion (FIG. 2—Fluo-4 channel).

The photographs and associated counts indicate a fusion efficiency above 80%, that is, more than 80% of all cells fused with the mixture/liposome according to the invention.

As a control, HEK293 cells were treated in an identical manner, except that the vesicular volume was filled with 20 mM HEPES instead of with 1 mM CaCl₂ buffer. In this case, the Ca²⁺ indicator indicated a much lower Ca²⁺ concentration in the cells (not shown).

Method 2 from FIG. 1: Cell membrane modification by mixing types A-C and incorporation of bacteriorhodopsin (molecule type E) into the cell membrane: Cardiac fibroblasts were isolated from embryonal (day 19) rat heart and incubated in F10 Ham's medium (Sigma Aldrich, St. Luis, Mo., USA) at a density of 20,000 cells per cell culture dish (Ø3.5 cm) at 37° C. and 5% CO₂.

Over a period of 6 days, the cardiac fibroblasts differentiated into myofibroblasts and were used in the following method according to the invention.

The myofibroblasts were incubated in a fusion mixture of 1,2-dioleoyl-sn-glycero-3-phosphoethanolamine (DOPE: molecule type C), 1,2-dioleoyl-3-trimethylammonium-propane (DOTAP: molecule type A) (both from Avanti Polar Lipids Inc. Alabama, Ala., USA) and 2-(4,4-difluoro-5-methyl-4-bora-3a,4a-diaza-s-indacene-3-dodecanoyl)-1-hexadecanoyl-sn-glycero-3-phosphocholine (β-BODIPY C₁₂-HPC: molecule type B from Invitrogen (Eugene, Oreg., USA) at a ratio by weight of 1/1/0.1 at a final concentration of 20 μg lipid/ml medium and fluorescence-marked channel protein (bacteriorhodopsin from Halobacterium Salinarum (Sigma Aldrich) tetramethylrhodamine isothiocyanate (Sigma Aldrich)) (BR-TRITC: molecule type E) 0.4 ng/ml).

The mixture according to the invention was prepared in the following manner. The lipid components (molecule type A-C) were mixed homogeneously in chloroform (VWR). After mixing, the solution was dried in a vacuum for 30-60 minutes. The lipid molecules were taken up once more in a buffer solution of 20 mM HEPES (2-[4-(2-hydroxyethyl)-1-piperazinyl]-ethanesulphonic acid) (VWR)) at a final concentration of 2 mg lipid/ml buffer and homogenized in an ultrasonic bath (80-100 W) for 20 minutes. All steps were carried out at room temperature. The mixture is therefore in the form of a liposome.

5 μl of this fusion mixture were incubated with 0.7 μl of a BR-TRITC solution (molecule type E) (0.06 mg/ml HEPES) for 60 minutes, while stirring carefully. The channel proteins E are taken up in the lipid bilayers of the liposome.

The solution was diluted 100-fold with F10 Ham's medium (Sigma Aldrich) and treated once more for 1 to 2 minutes with ultrasound (80-100 W) before being added to a cell culture dish containing myofibroblasts.

The cells were incubated according to the invention with the fusion mixture for 10 to 20 minutes, then washed with F10 Ham's medium and analyzed.

After washing, cytoplasmic membranes of the cells were detected in approximately 80 to 100% of the cells under the fluorescence microscope (green for lipids, red for proteins) (LSM 710 from Carl Zeiss Microimaging GmbH, Jena), thereby indicating successful incorporation into the membrane of the lipid mixture (molecule types A-C) and the protein molecules (molecule type E) (FIG. 3 and FIG. 4).

Method 3 from FIG. 1: Cell membrane modification by mixing types A-D for protein binding on the cell membrane surface, fundamentals of a new tumor treatment and a drug therefor.

Human embryonic kidney (HEK 293) (DSMZ, Germany) cells were incubated in RPMI medium (Sigma Aldrich, St. Luis, Mo., USA) at a cell density of 40,000 cells per cell culture dish (Ø3.5 cm) at 37° C. and 5% CO₂ and used at a confluent density of approximately 90% in the following step. HEK293 cells were incubated directly in a fusion mixture comprising 1,2-dioleoyl-sn-glycero-3-phosphoethanolamine (DOPE: molecule type C), 1,2-dioleoyl-3-trimethylammonium-propane (DOTAP: molecule type A), (both from Avanti Polar Lipids Inc. Alabama, Ala., USA), N-(4,4-difluoro-5,7-dimethyl-4-bora-3a,4a-diaza-s-indacene-3-propionyl)-1,2-dihexadecanoyl-sn-glycero-3-phosphoethanolamine, triethylammonium salt (Bodipy FL-DHPE: molecule type B) (Invitrogen, Eugene, Oreg., USA) and 1,2-dioleoyl-sn-glycero-3-[(N-(5-amino-1-carboxypentyl)iminodiacetic acid)succinyl] (nickel salt) (DOGS-NTA: molecule type D Avanti Polar Lipids Inc.) at a ratio by weight of DOPE/DOTAP/Bodipy FL-DHPE/DOGS-NTA (1/1/0.1/0.002 wt/wt) at a final concentration of 20 μg lipid/ml RPMI medium (Sigma Aldrich) for 10 to 20 minutes at 37° C. and 5% CO₂.

The fusion mixture was prepared in the following manner. The lipid components (molecule types A to D) were first mixed homogeneously in chloroform (VWR, Darmstadt, Germany). The solution was then dried in a vacuum for 30 to 60 minutes. The lipid molecules were taken up once more in a buffer solution of 20 mM (2-[4-(2-hydroxyethyl)-1-piperazinyl]-ethanesulphonic acid) (HEPES buffer) (VWR) at a final concentration of 2 mg lipid/ml buffer and homogenized in an ultrasonic bath (80-100 W) for 20 minutes.

The mixture is therefore in the form of a liposome. This mixture is stable for approximately 1 month under refrigeration at 4° C. and can be reused after re-homogenization.

5 μl of this fusion mixture were diluted 100-fold with RPMI medium (Sigma Aldrich) and re-homogenized for 5 to 10 minutes using ultrasound (80-100 W) before being added to a cell culture dish containing HEK293 cells. All preparation steps were carried out at room temperature. The cells were then washed with RPMI medium (Sigma Aldrich) and the fusion of the reagent with the cell membrane was examined using a fluorescence microscope (LSM 710 from Carl Zeiss Microimaging GmbH, Jena).

FIG. 5 shows an examination of this type and indicates a fusion efficiency above 80%. This efficiency was determined by counting using the microscopic photographs.

So as to allow binding to take place between the reactive head group of the DOGS-NTA used as molecule type D with a reaction partner, the cells were incubated in a solution of tumor necrosis factor-α bound to a 6× histidine repeat (TNFα-His-Tag) (ProSpec, Rehovot, Israel) at a concentration of 1 to 5 ng/ml of RPMI medium for approximately 20 minutes at 37° and 5% CO₂.

Protein residue was removed from the suspension by washing three times with RPMI medium. TNFα is used in the body as a macrophage-activating factor and is the main factor in the immune system for detecting and fighting cancer cells. Differentiated macrophages were used to check the functionality of the system. Human monocytes were obtained from blood using the Leukoseptsystem (Greiner bio-one, Kremsmünster, AU) and Biocoll separation medium (Biochrom, Cambridge, UK). After centrifugation at 1000 g for 10 minutes, monocytes were harvested from the interphase that formed, and were subsequently washed with phosphate buffer (PBS from Sigma Aldrich). Monocytes were taken up in RPMI medium (Sigma Aldrich) and incubated at 37° C. and 5% CO₂. After 2 days, 0.1 ng/ml granulocyte macrophage colony stimulation factor (G-MCF from Sigma Aldrich) was added to the monocytes. After another 3 days, inactive macrophages become differentiated from monocytes.

The macrophages obtained in this manner were then plated out on a HEK 293 cell sheet, which should carry the detection signal for macrophages by way of the fusion and subsequent incubation with TNFα-His-Tag. Upon detection, the cellular immune response should be induced in macrophages and HEK 293 cells should be lysed. As a control, HEK cells were treated in an identical manner except that the amphipathic molecule with the reactive head group of molecule type D (DOGS-NTA) was omitted from the fusion batch. Both batches were incubated for 24 hours and then analyzed using light-optical microscopy (LSM 710 from Carl Zeiss Microimaging GmbH, Jena). FIG. 6 shows the detection and lysis of HEK293 cells (formation of plaques) only on the cells incubated with DOGS-NTA (molecule type D) in the fusion batch. This example shows that, by way of membrane fusion, cell types can be marked in a specific manner and made visible to the immune system, for example.

A further exemplary embodiment of cell membrane modification by mixing types A-D for protein binding on the cell membrane surface by way of biotin-avidin/streptavidin binding:

Cardiac fibroblasts were isolated from embryonal (day 19) rat heart and incubated in F10 Ham's medium (Sigma Aldrich, St. Luis, Mo., USA) at a density of 20,000 cells per cell culture dish (Ø3.5 cm) at 37° C. and 5% CO₂. Over a period of 6 days, the cardiac fibroblasts differentiated into myofibroblasts and were used in the following method according to the invention.

The myofibroblasts were incubated directly in a fusion mixture of 1,2-dioleoyl-sn-glycero-3-phosphoethanolamine (DOPE: molecule type C), 1,2-dioleoyl-3-trimethylammonium-propane (DOTAP: molecule type A), (both from Avanti Polar Lipids Inc. Alabama, Ala., USA), Texas Red 1,2-dihexadecanoyl-sn-glycero-3-phosphoethanolamine (triethylammonium salt) (TexasRed DHPE: molecule type B) (Invitrogen, Eugene, Oreg., USA) and 1,2-dioleoyl-sn-glycero-3-phosphoethanolamine-N-cap-biotin (capBioDOPE: molecule type D Avanti Polar Lipids Inc.) at a ratio by weight of DOPE/DOTAP/TexasRedDHPE/capBioDOPE (1/1/0.1/0.1 wt/wt) at a final concentration of 20 μg lipid/ml F10 Ham's Medium (Sigma Aldrich) for 10 to 20 minutes at 37° C. and 5% CO₂.

The fusion mixture was prepared in the following manner. The lipid components (molecule types A to D) were first mixed homogeneously in chloroform (VWR, Darmstadt, Germany). The solution was then dried in a vacuum for 30 to 60 minutes. The lipid molecules were taken up once more in a buffer solution of 20 mM (2-[4-(2-hydroxyethyl)-1-piperazinyl]-ethanesulphonic acid) (HEPES buffer) (VWR) at a final concentration of 2 mg lipid/ml buffer and homogenized in an ultrasonic bath (80-100 W) for 20 minutes.

The mixture is therefore in the form of a liposome. This mixture is stable for approximately 1 month under refrigeration at 4° C. and can be reused after re-homogenization.

5 μl of this fusion mixture were diluted 100-fold with F10 Ham's Medium (Sigma Aldrich) and re-homogenized for 5 to 10 minutes using ultrasound (80-100 W) before being added to a cell culture dish containing myofibroblasts. All preparation steps were carried out at room temperature.

The cells were then washed with F10 Ham's Medium (Sigma Aldrich) and the fusion of the reagent with the cell membrane was examined using a fluorescence microscope (LSM 710 from Carl Zeiss Microimaging GmbH, Jena).

FIG. 7 shows an examination of this type and indicates a fusion efficiency above 80%. This efficiency was determined by counting using the microscopic photographs.

To enable binding to take place between the reactive head group of the capBioDOPE used as molecule type D with a reaction partner, the cells were incubated in a solution of streptavidin/streptavidin-AlexaFluoro488 (both from Invitrogen, Eugene, Oreg., USA) 100/1 mol/mol % in the total protein concentration of 1 mg/ml F10 Ham's medium for approximately 20 minutes at 37° C. and 5% CO₂.

Protein residue was removed from the suspension by washing three times with F10 Ham's Medium medium.

FIG. 7 shows unambiguous co-localization of phospholipid (red) and proteins bound to the cell surface (green).

A further exemplary embodiment of cell membrane modification by mixing types A-D to modify the cell membrane composition:

Cardiac fibroblasts were isolated from embryonal (day 19) rat heart and incubated in F10 Ham's medium (Sigma Aldrich, St. Luis, Mo., USA) at a density of 20,000 cells per cell culture dish (Ø3.5 cm) at 37° C. and 5% CO₂. Over a period of 6 days, the cardiac fibroblasts differentiated into myofibroblasts and were used in the following method according to the invention.

The myofibroblasts were incubated directly in a fusion mixture of 1,2-dioleoyl-sn-glycero-3-phosphoethanolamine (DOPE: molecule type C), 1,2-dioleoyl-3-trimethylammonium-propane (DOTAP: molecule type A), (both from Avanti Polar Lipids Inc. Alabama, Ala., USA), Texas Red 1,2-dihexadecanoyl-sn-glycero-3-phosphoethanolamine (triethylammonium salt) (TexasRed DHPE: molecule type B) and N-(4,4-difluoro-5,7-dimethyl-4-bora3a,4a-diaza-s-indacene-3-dodecanoyl)sphingosyl phosphocholine (B FL-SM: molecule type D) (both from Invitrogen, Eugene, Oreg., USA) at a ratio by weight of DOPE/DOTAP/TexasRed-DHPE/BFL-SM (1/1/0.1/0.01 wt/wt) at a final concentration of 20 μg lipid/ml F10 Ham's Medium (Sigma Aldrich) for 10 minutes at 37° C. and 5% CO₂.

The fusion mixture was prepared in the following manner. The lipid components (molecule types A to D) were first mixed homogeneously in chloroform (VWR, Darmstadt, Germany). The solution was then dried in a vacuum for 30 to 60 minutes. The lipid molecules were taken up once more in a buffer solution of 20 mM (2-[4-(2-hydroxyethyl)-1-piperazinyl]-ethanesulphonic acid) (HEPES buffer) (VWR) at a final concentration of 2 mg lipid/ml buffer and homogenized in an ultrasonic bath (80-100 W) for 20 minutes.

The mixture is therefore in the form of a liposome. This mixture is stable for approximately 1 month under refrigeration at 4° C. and can be reused after re-homogenization.

5 μl of this fusion mixture were diluted 100-fold with F10 Ham's Medium (Sigma Aldrich) and re-homogenized for 5 to 10 minutes using ultrasound (80-100 W) before being added to a cell culture dish containing myofibroblasts. All preparation steps were carried out at room temperature.

The cells were then washed with F10 Ham's Medium (Sigma Aldrich) and the fusion of the reagent with the cell membrane was examined using a fluorescence microscope (LSM 710 from Carl Zeiss Microimaging GmbH, Jena).

FIG. 8 shows an examination of this type within the scope of the exemplary embodiment and the incorporation of the phospholipid molecules (TexasRed DHPE) and the sphingolipids (BFL-SM) into the plasma membrane of a myofibroblast.

Method 4: Cell membrane modification by mixing types A-C on tissue samples:

Using the fusion mixture, fusion can be induced not only in single mammalian cells, but also in cells in tissue and tissue sections (see FIG. 9). Primary heart sac tissue (pericardium) was isolated from embryonal rat (day 19) and incubated in F10 Ham's medium (Sigma Aldrich, St. Luis, Mo., USA) on a 3.5 cm cell culture dish with a fusion mixture of 1,2-dioleoyl-sn-glycero-3-phosphoethanolamine (DOPE: molecule type C), 1,2-dioleoyl-3-trimethylammonium-propane (DOTAP: molecule type A), (both from Avanti Polar Lipids Inc. Alabama, Ala., USA) and N-(4,4-difluoro-5,7-dimethyl-4-bora-3a,4a-diaza-s-indacene-3-propionyl)-1,2-dihexadecanoyl-sn-glycero-3-phosphoethanolamine, triethylammonium salt (Bodipy FL-DHPE: molecule type B) (Invitrogen, Eugene, Oreg., USA) at a ratio by weight of 1/1/0.1 wt/wt at 37° C. and 5% CO₂ for 1 hour. (Molecule types C and B were obtained from Avanti Polar Lipids Inc., Alabama, Ala., USA; molecule type B was obtained from Invitrogen, Eugen, Oreg., USA). The fusion mixture was prepared in the following manner:

The lipid components (molecule types A to C) were first mixed homogeneously in chloroform (VWR, Darmstadt, Germany). The solution was then dried in a vacuum for 30 to 60 minutes. The lipid molecules were taken up once more in a buffer solution of 20 mM (2-[4-(2-hydroxyethyl)-1-piperazinyl]-ethanesulphonic acid) (HEPES buffer) (VWR, Darmstadt, Germany) at a final concentration of 2 mg lipid/ml buffer and homogenized in an ultrasonic bath (80-100 W) for 20 minutes. The mixture is in the form of a liposome which is stable at 4° C.

5 μl of this fusion mixture were diluted 100-fold with F10 Ham's medium (Sigma Aldrich) and re-homogenized for 5-10 minutes using ultrasound (80-100 W) before being added to the tissue sample. All preparation steps were carried out at room temperature.

The fused tissue was then analyzed using fluorescence microscopy (LSM 710 from Carl Zeiss Microimaging GmbH, Jena). FIG. 9 shows that cells of the outer tissue layer and cells in deeper tissue layers can be successfully fused and stained. Approximately 3-4 layers of connective tissue cells are clearly detectable. Potential applications in gene therapy, cancer treatment and wound healing are therefore feasible.

Further exemplary embodiments can be indicated. They are shown in Table 2. The methods for producing the mixtures are found in the four procedures provided herein as examples. Cell membrane fusion is performed according to these above-mentioned methods. Within the scope of the invention, all method steps in the exemplary embodiments should be considered non-restrictive in nature. In particular, the fusion mixtures and the use thereof should be broadly interpreted. This means that the methods and fusion mixtures shown in the exemplary embodiments can be combined with one another and result in new mixtures and embodiments.

The four methods presented herein in detail are based on liposomes since, after addition of chloroform and take-up in buffer, the amphipathic components are transformed into liposomes.

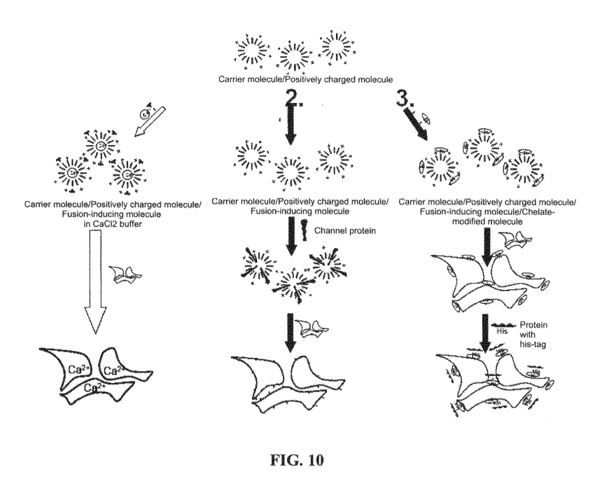

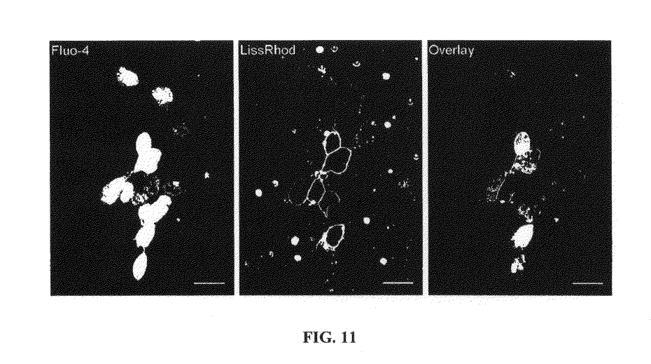

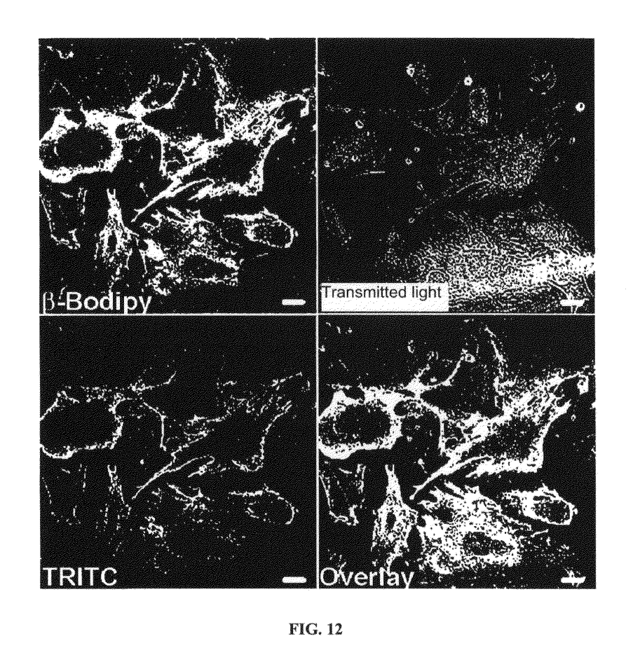

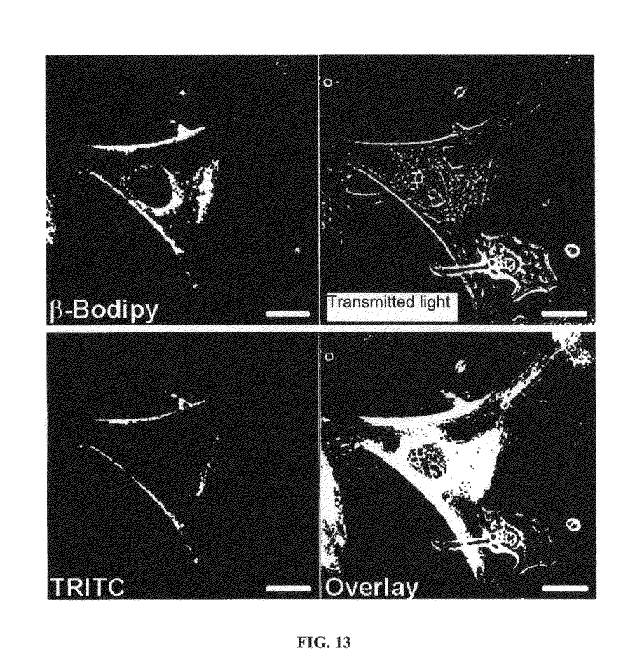

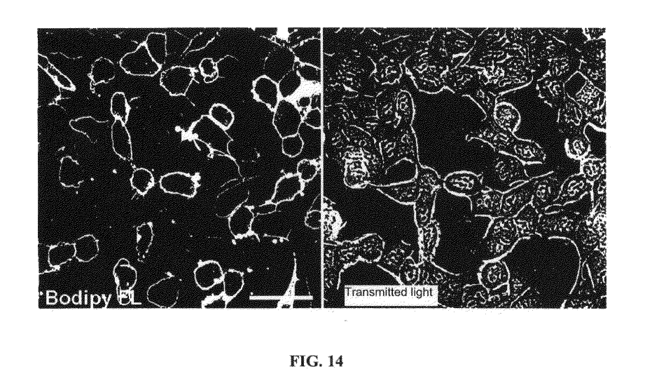

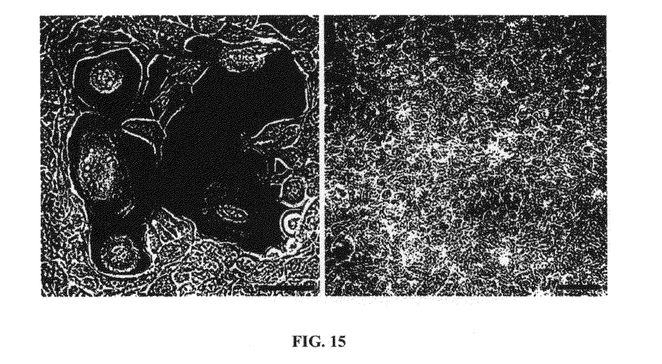

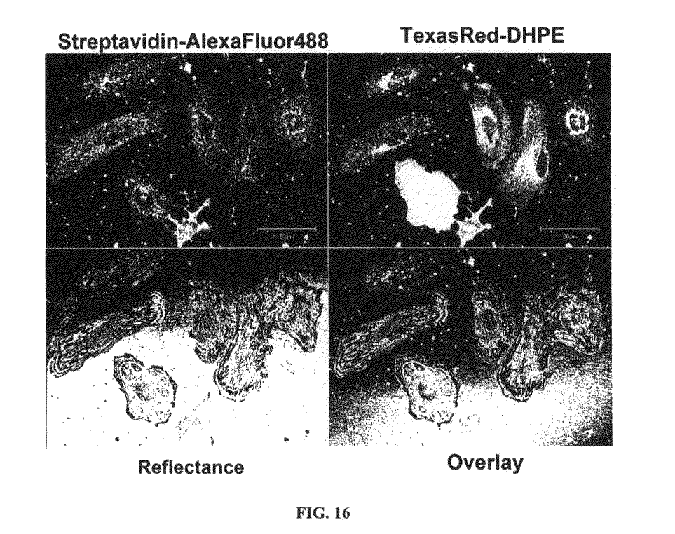

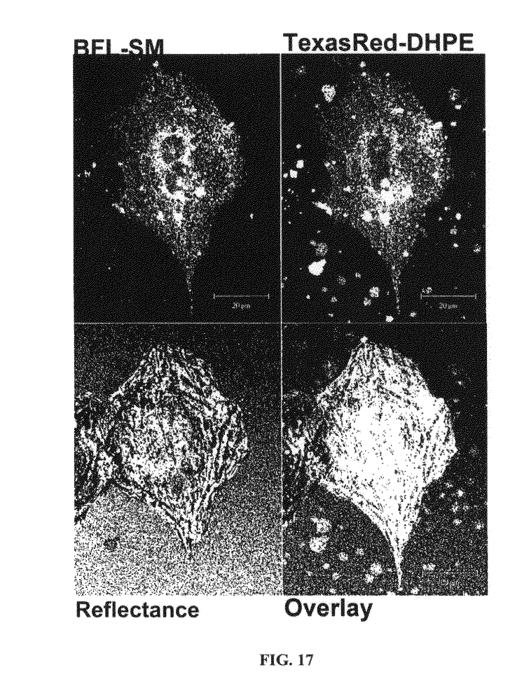

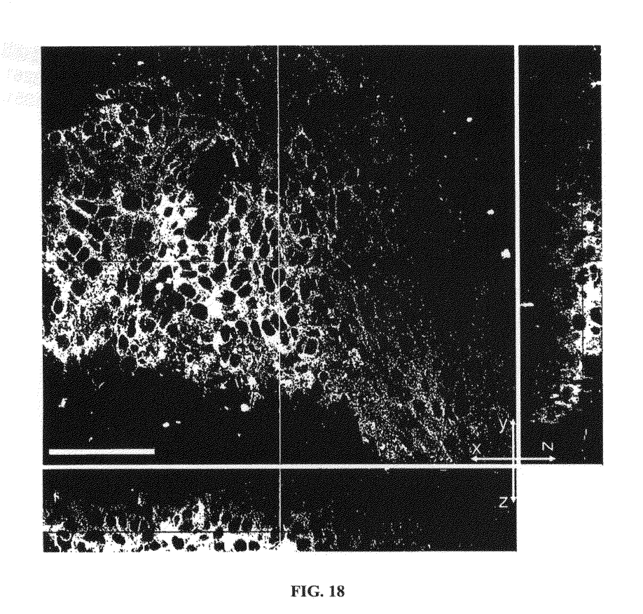

In the patent application, reference is made to colored FIGS. 1-9 attached to this patent application. Copies of these figures in black-and-white are attached to the patent application as FIGS. 10-18 in view of the disclosure requirements. The contents of FIG. 10 correspond to those of FIG. 1. FIG. 11 corresponds to FIG. 2. FIG. 12 corresponds to FIG. 3. FIG. 13 corresponds to FIG. 4. FIG. 14 corresponds to FIG. 5. FIG. 15 corresponds to FIG. 6. FIG. 16 corresponds to FIG. 7. FIG. 17 corresponds to FIG. 8. FIG. 18 corresponds to FIG. 9.

1. A mixture of amphipathic molecules comprising an amphipathic molecule type A which has a positive total charge in the hydrophilic region, and an amphipathic molecule type B which forms a delocalized electron system in the hydrophilic region and/or in the hydrophobic region.

2. The mixture according to claim 1, comprising a molecule type B having a delocalized electron system, comprising at least one cyclic structural motif formed by conjugated double bonds and/or free electron pairs and/or unoccupied p orbitals.

3. The mixture according to claim 1, wherein covalently bound adjacent atoms of molecule type B in the delocalized electron system have an electronegativity difference (Δ_{χ}) greater than or equal to 0.4.

4. The mixture according to claim 1, comprising a molecule type B comprising a group R which forms the delocalized electron system.

5. The mixture according to claim 1, comprising 1,2-dioleoyl-3-trimethylammonium-propane (DOTAP) as molecule type A.

6. The mixture according to claim 1, comprising a phospholipid, to which is bound a fluorescent dye having a delocalized electron system as the R group, as molecule type B.

7. The mixture according to claim 1, comprising 1-hexadecanoyl-2-dodecanoyl-sn-glycero-3-phosphocholine or 1,2-dihexadecanoyl-sn-glycero-3-phosphoethanolamine, or 1,2-dioleoyl-sn-glycero-3-phosphoethanolamine, as the phospholipid of molecule type B.

8. The mixture according to claim 1, comprising 2-(4,4-difluoro-5-methyl-4-bora-3a,4a-diaza-s-indacene-3-dodecanoyl) or N-(4,4-difluoro-5,7-dimethyl-4-bora-3a,4a-diaza-s-indacene-3-propionyl) or Lissamine rhodamine B sulfonyl or (5-(chlorosulfonyl)-2-(1H,2H,3H,5H,6H,7H,11H,12H,13H,15H,16H,17H-pyrido[3,2,1-ij]quinolizino[1′,9′:6,7,8]chromeno[2,3-f]quinolin-4-ium-9-yl)benzenesulfonate or 7-nitro-2-1,3-benzoxadiazol-4-yl or carboxyfluorescein or 1-pyrenesulfonyl which forms the delocalized electron system in molecule type B.

9. The mixture according to claim 1, comprising a further amphipathic molecule type C as a helper molecule.

10. The mixture according to claim 1, comprising 1,2-dioleoyl-sn-glycero-3-phosphoethanolamine (DOPE) as molecule type C.

11. The mixture according to claim 1, comprising a ratio of 1:0.05-0.2:1 between molecule types A, B and C.

12. The mixture according to claim 1 comprising up to 99% by weight of amphipathic molecule type A.

13. The mixture according to claim 1, comprising up to 40% by weight of amphipathic molecule type B.

14. The mixture according to claim 1, comprising up to 70% by weight of amphipathic molecule type C.

15. The mixture according to claim 1, wherein it is present dissolved in an aqueous buffer.

16. The mixture according to claim 1, comprising lipids as molecule type A and/or B and/or C.

17. A liposome comprising a mixture according to claim 15.

18. The mixture according to claim 1, comprising polymers as molecule type A and/or B and/or C.

19. A method for in vivo cell modification, comprising bringing into contact and fusing the cell membrane with a mixture or a liposome according claim 1.

20. The method according to the claim 19, comprising effecting contact between the cell membrane of a cell and the mixture during fusion of, at most, 1 to 120 minutes, and preferably 10 to 30 minutes.

21. The method according to claim 19, wherein a portion of cells fused with the mixture or the liposome (efficiency) of above 50%.

22. The method according to claim 19, wherein a functional group in the hydrophilic region of an amphipathic molecule type D, and in particular a chelate group functional group, is introduced to the mixture and is located on the surface of the cell during fusion.

23. The method according to claim 19, wherein a mixture comprising 1,2-dioleoyl-3-trimethylammonium propane is used as molecule type A and N-(4,4-difluoro-5,7-dimethyl-4-bora-3a,4a-diaza-s-indacene-3-propionyl)-1,2-dihexadecanoyl-sn-glycero-3-phosphoethanolamine is used as molecule type B, and triethylammonium salt/1,2-dioleoyl-sn-glycero-3-phosphoethanolamine is used as molecule type C, and 1,2-dioleoyl-sn-glycero-3-[(N-(5-amino-1-carboxypentyl)iminodiacetic acid)succinyl] (nickel salt) is used as molecule type D, for example at a mixing ratio of 1:0.1:1:0.002 wt/wt for fusion.

24. The method according to claim 23, wherein a further cell modification takes place by adding tumor necrosis factor (TNF)α bound to a 6× histidine repeat.

25. The method according to claim 19, wherein an amphipathic molecule type E is added to the mixture and is located in the membrane of the cell during fusion, in particular bacteriorhodopsin or glycophorin A or integrin as molecule type E.

26. The method according to claim 19, wherein a non-amphipathic, hydrophilic molecule type F is added in a buffered solution of a dried mixture and is delivered into the lumen of the cell during fusion, in particular with RNA, DNA, Ca²⁺, peptide, protein or a pharmaceutical such as acetylsalicylic acid as molecule type F.

27. The method according to claim 19, wherein a non-amphipathic, hydrophobic molecule type G is added to the mixture and is located in the membrane of the cell during fusion, in particular cholesterol, vitamin A, D, E and K or a pharmaceutical such as cortisone as molecule type G.

28. The method according to claim 1, comprising 1,2-dioleoyl-3-trimethylammonium-propane as molecule type A and N-(4,4-difluoro-5,7-dimethyl-4-bora-3a,4a-diaza-s-indacene-3-propionyl)1-dihexadecanoyl-sn-glycero-3-phosphoethanolamine as molecule type B, and triethylammonium salt/1,2-dioleoyl-sn-glycero-3-phosphoethanolamine as molecule type C, and 1-dioleoyl-sn-glycero-3-[(N-(5-amino-1-carboxypentyl)iminodiacetic acid)succinyl](nickel salt) as molecule type D, in particular in a mixing ratio of 1:0.1:1:0.002 wt/wt.

29. Cells fused in vivo with a mixture or a liposome according claim 1.
